SEQUENCE LISTING (4 sequences)

SEQ ID NO: 1 - protein, 71 aa, Naja kaouthia
ICRFITPDITSKDCPNGHVCYTKTWCDAFCSIRGKRVDLGCAATCPTVKTGVDIQCCSTDNCNPFPTRKRP

SEQ ID NO: 2 - protein, 71 aa, Naja naja
ICRFITPDITSKDCPNGHVCYTKTWCDGFCSIRGKRVDLGCAATCPTVKTGVDIQCCSTDNCNPFPTRKRP

SEQ ID NO: 3 - protein, 71 aa, Naja naja
ICRFITPDITSKDCPNGHVCYTKTWCDGFCSIRGKRVDLGCAATCPTVRTGVDIQCCSTDNCNPFPTRKRP

SEQ ID NO: 4 - protein, 71 aa, Naja naja
ICRFITPDITSKDCPNGHVCYTKTWCDGFCSSRGKRVDLGCAATCPTVRTGVDIQCCSTDNCNPFPTRKRP

(19) United States
(12) Patent Application Publication
(10) Pub. No.: US 2009/0209468 A1
(43) Pub. Date: Aug. 20, 2009

(54) Alpha-neurotoxin proteins with anti-inflammatory properties and uses thereof

(76) Inventors: Paul F. Reid, Sunrise, FL (US); Zheng-Hong Qin, Wilmington, MA (US)

Correspondence Address: Margaret J. McLaren, P.A.;McLaren Legal Services, 6500 S.W. 133rd Drive, Miami, FL, 33156, US

(21) Appl. No.: 12/317,115

(22) Filed: Dec. 19, 2008

Related U.S. Application Data
(63) Continuation-in-part of application No. 11/642,312, filed on Dec. 19, 2006.

(51) Int. Cl. A61K 38/16; A61P 19/02

(52) U.S. Cl. 514/12

(57) ABSTRACT

The invention provides methods and compositions for treating arthritic conditions such as osteoporosis and rheumatoid arthritis. The treatment methods include administering an effective amount of a pharmaceutical composition comprising an isolated alpha-neurotoxin protein, or an effective variant or fragment thereof. The compositions are effective for decreasing the levels of pro-inflammatory cytokines and increasing the level of interleukin-10 in a subject with arthritis, and can reduce symptoms of the arthritic condition including edema, infiltration of inflammatory cells and pannus formation in affected joints. In some preferred embodiments of compositions in accordance with the invention, the effective therapeutic protein is an alpha-neurotoxin protein isolated from snake venom, or a recombinant or synthetic protein based on, or derived from, the amino acid of sequence of an alpha-neurotoxin protein isolated from snake venom. Some preferred alpha-neurotoxin proteins are derived from the venom of elapid snakes including Naja naja and Naja kaouthia.

### CROSS-REFERENCE TO RELATED APPLICATIONS

The present application is a continuation-in-part of co-pending U.S. application Ser. No. 11/642,312, filed Dec. 19, 2006, entitled “Use of Cobratoxin as an Analgesic,” the disclosure of which is herein incorporated by reference in its entirety.

### FIELD OF THE INVENTION

The present invention generally relates to compositions and methods for the treatment of arthritis and associated conditions. More particularly, the invention provides for the use of bioactive alpha-neurotoxin proteins for this purpose.

### BACKGROUND OF THE INVENTION

Arthritis is a painful condition involving inflammation of the joints. Osteoarthritis (OA) is the most common form of arthritis in Western populations (Jordan et al., Ann Rheum Dis. 62(12):1145-55, 2003). OA involving the knee joint, which is characterized clinically by pain and functional disability, is the leading cause of chronic disability among the elderly in the US. Risk factors for OA include age, gender, race, trauma, repetitive stress/joint overload, muscle weakness, and genetic factors.

Pathologically, OA involves focal loss of articular cartilage and marginal and central new bone formation as well as involvement of the synovium, capsule, ligaments, periarticular muscle, and sensory nerves. Although OA was once considered a non-inflammatory arthropathy, recent evidence from preclinical and clinical studies supports the involvement of inflammation and inflammatory mediators in the pathophysiology of OA (Pelletier et al., Arthritis Rheum 44(6):1237-47, 2001). Both chondrocytes and synovium in OA can produce proinflammatory cytokines, including IL-1β, which can alter cartilage homeostasis in favor of cartilage degradation. Proinflammatory cytokines appear to be a major factor in stimulating matrix metalloproteinase synthesis and other cartilage catabolic responses in OA. Thus, inflammation and inflammatory mediators may play a role in the joint destruction associated with OA.

Current treatment of OA includes non-medicinal therapy, medicinal therapy, and surgical treatments. Non-medicinal treatments include exercise, thermal treatment, and assistive devices or bracing. Range-of-motion and strengthening exercises are geared toward reduction of impairment, improvement of function, and joint protection. Medications include analgesics (e.g., acetaminophen), non-steroidal anti-inflammatory drugs (NSAIDS) that are either non-selective cyclo-oxygenase (COX) inhibitors or selective inhibitors of the COX-2 enzyme, injected intra-articular corticosteroids or viscosupplementation, and proven or putative disease-modifying osteoarthritis drugs (DMOADs). Surgical procedures include joint debridement and lavage, and lastly, in the case of OA affecting the knee, total knee arthroplasty.

Another debilitating form of arthritis is Rheumatoid Arthritis (RA), including juvenile RA. RA is a systemic inflammatory disorder that affects approximately 1% of the population worldwide. According to the Arthritis Foundation, RA affects about 1.3 million adults and children in the U.S. alone. The disease is characterized by joint swelling, synovial membrane inflammation, cartilage destruction and erosion of bone. There is recognition that the spectrum of RA and the disease progression among individuals are governed by several factors including immune, genetic and environmental factors [1, 2]. Multiple components of immunity and inflammation are known to play a role in the onset and development of the disease, including T and B lymphocytes, neutrophils, monocytes, and vascular endothelium.

Results of recent research studies have identified certain so-called proinflammatory cytokines, such as tumor necrosis factor-α (TNF-α) and interleukin-1 (IL-1) as playing an important role in the pathology of various forms of arthritis. For instance, these cytokines have been shown to be increased in the synovial tissue, synovial fluid and serum in RA patients. In RA patients and in animal models of osteoarthritis and RA, IL-1 and TNF-α are known to promote abnormal proliferation of synoviocytes in affected joints and to increase the production of tissue enzymes such as matrix metalloproteinases by chondrocytes and synovial cells, resulting in cartilage degradation [3-5]. In the process of bone erosion, TNF-α triggers the production of other cytokines, induces endothelial adhesion molecules, stimulates collagenase and induces osteoclast differentiation [6]. Furthermore, TNF-α exerts its arthritogenic potency through the induction of IL-1. Increased expression of inflammatory cytokines including TNF-α and IL-1β has been observed in the bone of the knee joint and in serum samples from human patients with OA or rheumatoid arthritis [18]. It has also been demonstrated that TNF-α and IL-1β enhance the proliferation of fibroblasts, stimulate the production of prostaglandin E₂ (PGE₂) [19], and increase the expression of cytokines and synthesis of collagen by synovial cells, contributing to cartilage and bone destruction [20].

Based on such evidence, IL-1, IL-2 and TNF-α have become recognized as prominant molecular entities in the induction of inflammation and bone erosion that accompanies arthritis. Hence, these cytokines have been targets of drug discovery efforts [7, 8]. Various strategies to block the actions of the pro-inflammatory cytokines have been tested in pre-clinical studies using animal models of arthritis and in human clinical trials [21].

The development of new and improved strategies for inhibiting pro-inflammatory cytokines continues to holds promise as a therapeutic approach to treating arthritis. It would be particularly beneficial to develop an effective therapeutic agent for arthritis that could be efficiently and inexpensively produced, permitting its widespread availability to the growing population of arthritis sufferers around the globe.

### SUMMARY OF THE INVENTION

The invention provides methods and compositions for the treatment of arthritic conditions based on the use of a protein of the class known as an alpha-neurotoxin (α-neurotoxin), and in particular those α-neurotoxin proteins that can be derived from the venom of cobras (snakes of the genus Naja). The inventors have made the important discovery that α-cobratoxin (CTX), a long-chain α-neurotoxin that can be isolated, e.g., from the venom of the Thailand cobra, has a pronounced anti-inflammatory effect in an animal model of arthritis. More particularly, administration of the α-neurotoxin significantly ameliorates the symptoms of arthritis in an animal model characterized by pathophysiological hallmarks such as joint swelling, proliferation of synoviocytes, and joint inflammation associated histopathologically with infiltration of mononuclear cells into the sub-synovial tissue and accumulation of collagen.

Consistent with results of studies described above, the inventors have confirmed that the pathological changes in the arthritis model are accompanied by increased production of the pro-inflammatory cytokines TNF-α, IL-1, and IL-2. They have further shown that treatment of the arthritic animals with a long-chain α-neurotoxin protein in accordance with the invention, which ameliorates the pathiophysiological symptoms in the affected joints as described, is accompanied by a significantly reduced production of the pro-inflammatory cytokines TNF-α, IL-1, and IL-2, and an increase in production of the anti-inflammatory cytokine IL-10, as measured in the serum of these animals.

The efficacy of the treatment method for arthritic conditions is further demonstrated herein in a human subject diagnosed with rheumatoid arthritis who suffered from pain and reduced mobility in his hands. Use of a topical formulation of a pharmaceutical composition in accordance with the invention comprising α-cobratoxin protein in a suitable base effectively provided relief from this pain and increased the mobility in the subject's hands.

Based on these discoveries, the invention provides, in one aspect, a method of treating an arthritic condition comprising administering to a subject in need thereof a pharmaceutical composition comprising an isolated α-neurotoxin protein, or an effective variant or fragment thereof.

The treatment method is shown herein to be effective in reducing a variety of symptoms of arthritis in the subject. For example, the treatment can be effective for reducing edema in a joint of the subject. The treatment can be effective in reducing infiltration of inflammatory cells into articular cartilage in a joint of the subject, and in reducing pannus formation in an affected joint of the subject.

The treatment method can be effective in causing a decrease in the level of at least one pro-inflammatory cytokine in the serum of a subject with an arthritic condition such as OA or RA. In some preferred embodiments, the method of treatment is effective in reducing the serum levels of a pro-inflammatory cytokine, including the cytokines TNF-α, interleukin-1 (IL-1), and interleukin-2 (IL-2).

In some preferred embodiments, the treatment method is effective in increasing the level of the anti-inflammatory interleukin-10 (IL-10) in the serum of an arthritic subject.

Particularly preferred embodiments of the method of treatment utilize isolated alpha-neurotoxin proteins that can effect both an increase in IL-10 and a decrease in one or more pro-inflammatory cytokines in the serum of the subject.

In some preferred treatment methods in accordance with the invention, the isolated α-neurotoxin protein is derived from the venom of an elapid snake. Particularly preferred elapid α-neurotoxin proteins of use in the invention can be isolated, e.g., from the venom of the cobra species Naja kaouthia and Naja naja.

One particularly preferred embodiment of the treatment method utilizes an isolated α-neurotoxin protein that is a 71 amino acid protein known as a long-form α-cobratoxin protein. Particular examples of effective long form α-cobratoxin proteins have the amino acid sequence identified as any one of SEQ ID NOS: 1, 2, 3, or 4, or an effective variant or fragment thereof.

Some embodiments of the treatment method utilize effective proteins produced by recombinant DNA technology rather than proteins isolated from snake venom. Accordingly, the methods of the invention encompass the use of both isolated, naturally occurring α-neurotoxin proteins from snake venoms and corresponding and related proteins that are produced by recombinant DNA techniques.

In one preferred embodiment, the isolated α-neurotoxin protein is a recombinant long-form α-cobratoxin protein or an active fragment or variant thereof. The amino acid sequence of the recombinant protein can comprise the sequence identified as any one of SEQ ID NOS.: 1, 2, 3, or 4, or it can be an active variant or fragment of any one of these sequences.

In the practice of the method in accordance with the invention, the composition comprising the α-neurotoxin protein is administered at a dosage of from about 0.01 to 30 micrograms per kilogram of body weight, and preferably from about 0.75-8.0 micrograms per kilogram body weight.

Compositions in accordance with the invention can be administered orally, intravenously, intraperitoneally, topically, or nasally.

Particularly preferred applications of methods in accordance with the present invention include the treatment of subjects, and in particular human patients, suffering from rheumatoid arthritis and osteoarthritis.

### BRIEF DESCRIPTION OF THE DRAWINGS

FIGS. 1A and 1B are two graphs showing the effect of treating an arthritic condition using a composition comprising an α-neurotoxin protein (CTX) in accordance with an embodiment of the present invention. The graphs show reduction of swelling of the knee joint in an arthritic animal model (CFA-induced arthritis), following pre-treatment (1A) and post-treatment (1B) with the composition.

FIGS. 2A-D are four bar graphs showing, in control and CFA-induced arthritic (CFA) animals treated with CTX or saline, the effect on the serum levels of pro-inflammatory cytokines IL-1, TNF-α and IL-2 (FIGS. 2A, 2B, and 2C, respectively) and of the anti-inflammatory cytokine IL-10 (FIG. 2D). Arthritic groups receiving CTX treatment show a pattern of decrease in pro-inflammatory cytokines and an increase in IL-10 relative to the saline-treated arthritic group.

FIGS. 3A-F are six photomicrographs showing the histological appearance of sections from knee joints of arthritic animals under conditions of treatment with CTX, or saline control as described. The results show amelioration of pathological features with CTX treatment. Further details are provided infra in the accompanying description.

### DETAILED DESCRIPTION OF THE INVENTION

The invention relates to long chain alpha-neurotoxin (α-neurotoxin) proteins that are present among a heterogeneous mixture of biologically active proteins contained in the venoms of certain classes of snakes. It has now been discovered that certain α-neurotoxin proteins isolated and substantially purified from the venom of cobra (elapid) species such as Naja naja and Naja kaouthia among others, when administered at a low dosage, can exert beneficial immunomodulatory effects, and alleviate pathological symptoms in both an experimental model of arthritis and in human rheumatoid arthritis.

The terms “arthritic condition,” and “arthritis,” as used herein, are meant to include any of a variety of diseases and conditions that include or associated with inflammation and pain in the joints of an animal, including but not limited to osteoarthritis (OA), and adult and juvenile forms of rheumatoid arthritis (RS).

In various embodiments, the invention utilizes pharmaceutical compositions of matter comprising isolated, substantially purified or homogenous protein or polypeptide components, either derived from snake venom, or produced synthetically by recombinant DNA technology, (either as full length polypeptides based on the naturally occurring proteins, or effective variants or fragments of amino acid sequences derived from the naturally-occurring or recombinant proteins), as further described below.

Used in methods of treatment in accordance with the present invention, these therapeutic compositions provide a major advantage over previous approaches utilizing whole venoms, such as whole cobra venom as used in traditional Chinese medicine to treat arthritis, or compositions containing a wide mixture of proteins or polypeptides made by combining whole venoms or venom fractions obtained from a plurality of different snake genera, such as elapid, krait and vipered (see, for example U.S. Pat. No. 4,341,762 to Haast). Advantageously, as compared with the prior art venoms and combinations of fractions, the pharmaceutical compositions in accordance with the invention comprise as their active components an isolated therapeutic protein or polypeptide that is specifically known, and for which the physiological effects are testable in isolation and can be accurately attributed to a unique molecular entity. Thus, it is believed that pharmaceutical compositions in accordance with the invention, comprising an effective α-neurotoxin protein or polypeptide derived from snake venom and provided in a substantially purified form in isolation from other venom components, hold great promise for the treatment or prevention of arthritic conditions.

In one aspect, the present invention is directed to a method of treating arthritic conditions comprising administering to a subject in need thereof an effective amount of a pharmaceutical composition comprising an isolated α-neurotoxin protein, or an effective variant or fragment thereof.

The terms “isolated” and “substantially purified,” as the terms are used herein, are meant to refer to naturally occurring α-neurotoxin proteins and polypeptides of snake venoms that have been isolated from other protein and polypeptide species and from other classes of molecules that are contained in a venom mixture as it occurs in a snake venom gland or after it has been obtained from a snake (typically by a process known as “milking”). The terms “isolated” and “substantially purified” in this context can also refer to a recombinant or synthetic α-neurotoxin nucleic acid or polypeptide molecule that has been synthesized by man and separated from other molecules having different chemical structures or nucleic acid or amino acid sequences than the isolated α-neurotoxin molecule of interest.

As discussed, the active protein in the pharmaceutical compositions can be derived from the venom of snakes. Culture of snakes, and methods of obtaining their venoms, have been practiced since antiquity in many regions around the globe in which venomous snakes are indigenous. Typically, venom from a particular species of snake is milked, pooled with other such venoms from the same species and then dried, e.g., by lyophilization. Dried venom products from a variety of snake species are commercially produced in many countries. Such venom products can provide one abundant source of starting material from which to purify a desired α-neurotoxin protein.

One preferred genus of elapid snakes that can serve as a good source of α-neurotoxin venom proteins is the genus Naja. Currently, about 20 Naja species are recognized, and include N. annulifera (commonly known as snouted cobra), N. atra (Chinese cobra), N. haje (Egyptian cobra), N. kaouthia (monocled cobra), N. katiensis (Mali cobra), N. mandalayensis (Mandalay spitting cobra), N. melanoleuca (Black and white cobra, Forest cobra), N. mossambica (Mozambique spitting cobra), N. naja (Indian cobra), N. nigricollis (Black-necked spitting cobra), N. nivea (Cape cobra), N. nubiae (Nubian spitting cobra), N. oxiana (Central Asian cobra), N. pallida (Red spitting cobra), N. phillippinensis (Phillipine cobra), N. sagittifera (Andaman cobra), N. samarensis (Peter's cobra), N. siamensis (Indo-Chinese spitting cobra, Black and White spitting cobra), N. sputatrix (Indonesian cobra) and N. sumatrana (Golden spitting cobra).

Other preferred sources of long-chain alpha neurotoxins include the venoms of snakes such as Mambas (e.g., Dendroaspis polylepis, D. angusticeps, D. viridis, and D. jamesoni); King cobras (e.g., Ophiophagus hannah), Kraits (e.g., Bungarus multicintus), and Australian elapids (e.g., Notechis scutatus, and Acanthophis antarcticus).

Methods involving molecular biological, cell biological, conventional and analytical chemistry techniques are described herein. Such techniques are generally known in the art and are described in detail in methodology treatises. Molecular biological, biochemical and cell biological methods are described in treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Chemistry methods are described, e.g., in Classics in Total Synthesis. Targets, Strategies, Methods, K. C. Nicolaou and E. J. Sorensen, VCH, New York, 1996; The Logic of Chemical Synthesis, E. J. Coney and Xue-Min Cheng, Wiley & Sons, NY, 1989; and NMR of Proteins and Nucleic Acids, Wuthrich, K., Wiley & Sons, New York, 1986.

Methods of isolating and purifying proteins from complex biological mixtures are generally well known to those of skill in the art of biochemistry (see, e.g., Sambrook, supra). Particular methods useful for the isolation and purification of venom proteins are also known, and have been described, for example, in Miller K D et al., Biochim. Biophys. Acta 496:192-196, 1977. In general, one approach is to use traditional column chromatography to separate protein components from a whole venom preparation. Typically, a lyophilized venom such as a cobra venom is suspended in a suitable buffer and chromatographed, e.g., on a 2.5×20 cm column of SP-Sephadex, C-25, e.g., using a modification of the method of Chatman and DiMari, Toxicon 12:405-414, 1974, as described in Miller, supra. Use of this chromatographic method resolves cobra venom, e.g., from Naja kaouthia, into about 16 toxic and nontoxic fractions. The principal neurotoxin preparations are chromatographically and electrophoretically homogeneous within these fractions.

In general, identification of fractions containing neurotoxins is carried out by testing toxicity in mice. For example, mice are administered a 0.5 ml intraperitoneal injection of an equivalent amount (e.g., 5 μg of protein) from each of the chromatographic fractions. Injected mice are then monitored for the induction of respiratory paralysis, which is characteristic of cobra neurotoxin toxicity. A half lethal dose (LD₅₀) can be calculated, for example, according to the method of Reed and Muench, Am. J. Hygiene 27:493-497, 1938. As a non-limiting example, the isolation method of Miller et al., described above, results in chromatographic fractions designated as VIb and VIII containing, respectively, the invariant principal and minor neurotoxins of Naja siamensis venom as described by these investigators. Fraction VIb preparations are pure, as determined by disc electrophoresis and yield a half lethal dose (LD₅₀) of 1.1-1.5 μg per 20 g mouse.

An alternative approach to separation of native proteins from venom such as by the chromatographic procedures discussed above, or by other biochemical methods, involves synthetically preparing an α-neurotoxin protein. This approach involves the use of DNA (oligonucleotide) cloning and recombinant protein production technology. One such approach is described, e.g., in Antil S. et al., J. Biol. Chem. 274 (49):34851-58, 1999. In brief, this approach is based upon the knowledge of the primary amino acid sequence of a native α-neurotoxin protein to be synthesized. For example, as described by Antil et al., one approach to cloning the long chain toxin from Naja kaouthia, (α-cobratoxin (CTX), also termed “α-Cbtx”) and producing this protein in a bacterium such as E. coli can be performed as follows.

A synthetic gene for CTX can be designed by back-translating the primary amino acid sequence of native CTX protein and then optimizing the codon usage for expression in a chosen bacterium. In a typical synthesis, the desired synthetic gene can be made using sets (e.g. 5 sets) of purified synthetic oligomers ranging in length from about 25 to 60 nucleotides. Equimolar mixtures of sense and complementary oligoneucleotides are annealed, e.g., at 95° C. for 5 minutes, and then cooled to room temperature overnight. Using techniques well known in the art of molecular biology, the double-stranded oligonucleotides are then ligated, e.g., using the enzyme T4 DNA ligase, to form the complete synthetic gene having the desired coding sequence. Alternatively, using more modern methods, the entire sequence can be synthesized in one piece. The synthetic gene is preferably flanked on either end by restriction enzyme sites (such as KpnI and BamHI) to facilitate manipulation of the gene construct in various vectors.

The final gene construct encoding the desired α-neurotoxin protein can be amplified by standard polymerase chain reaction (PCR) procedures, using appropriately designed oligonucleotide primers. For example, the amplified PCR product can be isolated by low melting agarose electrophoresis and then digested with KpnI and BamHI enzymes, and ligated into a suitable vector. One effective strategy is to combine a ZZ fusion protein strategy with a pCP vector. A pCP vector is a pET3a derivative plasmid, as fully described in Drevet et al., Prot. Expression Purif. 10:293-300, 1997. In this approach, the doubly-digested fragment is ligated into a pCP plasmid vector that has been previously digested with KpnI and BamHI, e.g., for 3 hours at 16° C. The ligation mixture, thus prepared, is used to transform a suitable bacterial host cell, such as a DH5α-competent cell. Following transformation of the DH5α cells with the ligation mixture, the presence of the desired sequence is verified by preparing several (e.g., 5) isolated clones of cells, and sequencing the plasmid product by double-stranded DNA sequencing (for example, using a T7 Sequencing Kit from Amersham Pharmacia Biotech). Alternatively, DNA sequence analysis can be performed by any other suitable method known in the art.

One method to obtain optimal expression of the recombinant CTX protein utilizes a ZZ fusion protein that can be induced in the E. coli host strain BL21(DE3). This bacterial strain contains the structural gene for the T7 bacteriophage RNA polymerase under lac repressor control. Isolated plasmid comprising the desired coding sequence, described above, is used to transform the BL21(DE3) strain. In brief, to produce the desired fusion proteins, the BL21(DE3) bacteria are grown under suitable conditions, e.g., at 37° C. to late log phase (A₆₀₀=0.6 to 0.8) in media such as Luria broth (Difco) supplemented with 200 μg/ml ampicillin and 30 μg/ml chloramphenicol. The bacteria are then induced with a suitable inducer such as 1 mM isopropyl-1-β-D-thio-1-galactopyranoside (Eurobio) for 3 hr. Cells are centrifuged and resuspended in lysis buffer (e.g., 30 mM Tris, pH 8, 5 mM EDTA, 20% sucrose, 0.1 mg/ml lysozyme (Sigma) and 0.5 mM phenyl-methylsulfonyl fluoride (Sigma)). After three freezing-thawing steps, 2 mg/ml protamine sulfate (Sigma) is added, and the solution is centrifuged, e.g., at 12,000 rpm for 10 minutes. The supernatant is then applied to an IgG-Sepharose-6FF column (Amersham Pharmacia Biotech) and the fusin protein is eluted with 0.5M acetic acid, pH 3.4, and lyophilized. Protein concentration can be estimated by UV absorption at 280 nm.

Alternatively, any suitable protein expression system (e.g., a GST fusion protein system (Amersham Pharmacia Biotech)) can be utilized. All such systems include methods of removing isolation tags such as GST from fusion proteins, to yield as the final product an isolated, pure protein having the desired amino acid sequence of interest, free of the tag sequence.

Yet another approach to producing an α-neurotoxin protein or polypeptide in accordance with the present invention is to use recombinant DNA technology involving molecular cloning of a gene encoding a selected α-neurotoxin protein. For example, U.S. Pat. No. 6,670,148 describes a protocol for cloning the gene encoding α-cobratoxin from Naja naja siamensis (now known as Naja kaouthia), using techniques well known in the art of molecular biology. Briefly, using messenger RNA (mRNA) purified from total RNA extracted from venom glands surgically removed from the snakes, a N. n. siamensis cDNA library is prepared using a commercially available cDNA synthesis kit. The N. n. siamensis cDNA library is ligated into a suitable vector such as Lambda ZAPII (Stratagene) and packaged into viable bacteriophage particles. The recombinant bacteriophage are used to infect a suitable bacterial host strain, such as E. coli XL1-Blue, to generate the primary cDNA library. Following amplification of the primary cDNA library, plaques from the library are screened for sequences encoding the selected α-neurotoxin protein, using degenerate oligonucleotide probes prepared from the known amino acid sequence of the α-neurotoxin.

A wide variety of α-neurotoxin proteins and polypeptides can be isolated using the biochemical or recombinant methods as described above, and will find utility in methods of treatment of arthritic conditions in accordance with the invention. Many naturally-occurring protein and polypeptide molecules contained in snake venoms have been isolated and extensively studied. The predominent toxic components of venom have been classified as cardiotoxins and α-neurotoxins. Amino acid sequences have been determined for more than 80 cardio- and α-neurotoxin proteins, which fall into two groups. The cardiotoxins and the “short” neurotoxin proteins consist of 60-62 amino acids, whereas the “long” α-neurotoxin proteins consist of 70-74 amino acids (See, e.g., Endo and Tamija, Pharmacol. Ther. 34:403-451, 1987).

Alignment of the amino acid sequences of cardiotoxins and α-neurotoxin proteins reveals a number of conserved or conservatively changed residues, and either four or five disulfide bonds in identical positions (in short and long toxins, respectively), suggesting that the overall folding of the polypeptide chains are similar (Betzel C et al., J. Biol. Chem. 266:21530-21536, 1991). The similarity of structure among these proteins has been confirmed by X-ray crystallographic studies performed on several of these polypeptides, including: cardiotoxin V from Naja mossambica mossambica; the short erabutoxin b from Laticauda semifasciciata; α-bungarotoxin from Bungarus multicinctu; and two long toxins (α-cobratoxins) from Naja naja siamensis.

All of these molecules have been shown to have a comparable three-dimensional structure. The polypeptide chain of α-neurotoxin proteins is characterized by the being organized into three major loops which emerge like the fingers of a hand from a “palm” which is knotted together by the disulfide bonds. The central loop is more prominent than the other two. In the long α-neurotoxins, the central loop contains an extra disulfide bond at its lower tip and these toxins feature an additional “tail” at the C-terminus. The shapes of all of these toxin proteins are similar, resembling a hand or saucer, with the concave side harboring the amino acid side chains thought to be responsible for the toxic activity (Betzel et al., supra). In the crystal lattice, cardiotoxin, α-bungarotoxin and α-cobratoxin dimerize by β-sheet formation between residues 53 and 57 of symmetry-related molecules. This feature is thought to be of importance for interaction of these molecules with cellular receptors such as the acetylcholine receptor (NAchR) for example.

A particularly preferred class of long form α-neurotoxin protein in accordance with the present invention is an α-cobratoxin protein (abbreviated “CTX”) having 71 amino acid residues. This protein, which has been isolated and purified from the venom of Naja naja and Naja kaouthia, is shown herein to be effective in alleviating clinical symptoms of arthritic conditions when administered either intraperitoneally or in a topical pharmaceutical composition. Briefly, in pre-clinical studies, the CTX-containing composition was tested in an adjuvant-induced animal model of arthritis characterized by joint edema and accompanying histopathological changes including infiltration of inflammatory cells into the articular cartilage, proliferation of synoviocytes, and pannus formation. All of these symptoms were ameliorated by treatment of the animals by intraperitoneal injection of a physiological solution containing an effective amount of CTX. Determination of the levels of cytokines in the serum of these animals further demonstrated that the CTX treatment significantly decreased the levels of pro-inflammatory cytokines and increased the level of an anti-inflammatory cytokine in this model.

Additionally, in a trial in a human subject with rheumatoid arthritis, topical application of a pharmaceutical composition comprising CTX in a suitable (cream) base was effective in relieving joint pain and in increasing mobility in the hands of the subject in areas where the cream was applied. (See further details in Examples, infra). Accordingly, CTX is considered to be of significant therapeutic benefit in the treatment of arthritic conditions.

Table I provides a list of amino acid sequences of several exemplary therapeutically effective CTX proteins suitable for use in the invention.

TABLE 1

Amino Acid Sequences of Selected Alpha-cobratoxin
(CTX) Proteins

| | |
|---|---|
| ICRFITPDITSKDCPNGHVCYTKTWCDAFCSIRGKR | (SEQ ID NO. 1) |
| VDLGCAATCPTVKTGVDIQCCSTDNCNPFPTRKRP | |
| ICRFITPDITSKDCPNGHVCYTKTWCDGFCSIRGKR | (SEQ ID NO. 2) |
| VDLGCAATCPTVKTGVDIQCCSTDNCNPFPTRKRP | |
| ICRFITPDITSKDCPNGHVCYTKTWCDGFCSIRGKR | (SEQ ID NO. 3) |
| VDLGCAATCPTVRTGVDIQCCSTDNCNPFPTRKRP | |
| ICRFITPDITSKDCPNGHVCYTKTWCDGFCSSRGKR | (SEQ ID NO. 4) |
| VDLGCAATCPTVRTGVDIQCCSTDNCNPFPTRKRP | |

The sequence identified herein as SEQ ID NO. 1 is naturally occurring in the venom of Naja kaouthia. The sequences identified as SEQ ID NOS. 2-4 are found in native venom of Nana naja naja.

Other preferred sequences are effective fragments or variants of an α-neurotoxin protein sequence that demonstrate, e.g., at least 80% of the activity of the corresponding full-length protein, as determined by a standard bioactivity assay such as one or more of the tests of anti-arthritic activity as described in the Examples below.

Because modulation of inflammatory cytokines is central to a very large number of physiological processes in animals and man, the invention is believed to be compatible with any animal subject. A non-exhaustive list of examples of such animals includes mammals such as mice, rats, rabbits, goats, sheep, pigs, horses, cattle, dogs, cats, and primates such as monkeys, apes, and human beings. Those animal subjects having an arthritic disease or condition that relates to modulation of pro-inflammatory cytokine levels and/or IL-10 are preferred subjects, as these animals may have the symptoms of their disease reduced or even reversed. In particular, human patients suffering from inflammatory conditions such as osteoarthritis and rheumatoid arthritis might especially benefit.

The compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found, for example, in Remington's Pharmaceutical Sciences, a standard text in this field, and in USP/NF. Other substances may be added to the compositions to stabilize and/or preserve the compositions.

The compositions of the invention may be administered to animals by any conventional technique. Such administration may be oral or parenteral (for example, by intravenous, subcutaneous, intramuscular, or intraperitoneal introduction). The compositions may also be administered directly to the target site by, for example, surgical delivery to an internal or external target site, or by catheter to a site accessible by a blood vessel. Other methods of delivery, for example, liposomal delivery or diffusion from a device impregnated with the composition, are known in the art. The compositions may be administered in a single bolus, multiple injections, or by continuous infusion (for example, intravenously or by peritoneal dialysis). For parenteral administration, the compositions are preferably formulated in a sterilized pyrogen-free form.

Compositions in accordance with the present invention can also be administered in vitro to a cell (e.g., for testing effectiveness of a selected α-neurotoxin protein in an in vitro cell-based assay). Such an assay might be designed, e.g., to screen for modulation of cytokine production by the target cell such as an immune cell or cell type from a joint tissue, following exposure to the test protein, or to test selective binding of a test protein to a particular cell surface receptor such as a nicotinic acetylcholine receptor or other type of receptor of interest.

As is well known in the medical and veterinary arts, dosage for any one animal depends on many factors, including the particular animal's size, body surface area, age, the particular composition to be administered, time and route of administration, general health, and presence of other drugs being administered concurrently. It is expected that an appropriate dosage for parenteral or oral administration of α-neurotoxin compositions in accordance with the invention would be in the range of about 0.01 to 30 μg/kg, and preferably in the range of 0.75-8.0 μg/kg of body weight in humans. An effective amount for use with a cell in culture will also vary, but can be readily determined empirically (for example, by adding varying concentrations to the cell and selecting the concentration that best produces the desired result).

An “effective amount” is an amount of a pharmaceutical composition that is capable of producing a desirable result in a treated animal or cell. For example, in an animal host suffering from an arthritic condition, a desirable result can be a reduction or elimination of one or more symptoms of the disease, such as joint swelling, joint pain, infiltration of inflammatory cells, proliferation of synoviocytes or pannus formation in an affected joint. The desirable result may also be a reduction in the levels of pro-inflammatory cytokines such as TNF-α, IL-1 and IL-2, e.g., as measured in the serum of the host, and/or a desirable increase in serum level of an anti-inflammatory cytokine such as interleukin-10 (IL-10). Cyokine production levels can also be measured using isolated cells of the animal subject, or in a cell in culture).

Toxicity and efficacy of the compositions of the invention can be determined by standard pharmaceutical procedures, using cells in culture and/or experimental animals to determine the LD₅₀ (the dose lethal to 50% of the population) and the ED₅₀ (the dose that produces the desired result in 50% of the population). Compositions that exhibit a large LD₅₀/ED₅₀ ratio are preferred. Although less toxic compositions are generally preferred, more toxic compositions may sometimes be used in in vivo applications if appropriate steps are taken to minimize the toxic side effects. It is noteworthy that CTX is not overtly toxic to normal cells in culture.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

### EXAMPLES

The present invention is further described by reference to the following Examples, which are offered by way of illustration and are not intended to limit the invention in any manner. Standard techniques well known in the art, or the techniques specifically described below, were utilized.

### Example 1

### Materials and Methods

1.1 Experimental Animals. Male SD rats weighing 180-220 g were purchased from the Experimental Animal Center of Soochow University. The animals were fed ad libitum and were housed in a room with a controlled ambient temperature (22±2° C.), humidity (50±10%), and lighting (12-h light/dark cycle). Animals were acclimated to the housing conditions and handled for 3-4 days before experiments. All experiments were performed between 08.00 h and 16.00 h. All experimental procedures were conducted according to the NIH Guidelines for the Care and Use of Laboratory Animals (NIH Publications No. 80-23, revised 1996). The experimental protocols were approved by the Local Committee on Animal Care and Use at Soochow University.

1.2 Reagents. Cobratoxin (CTX), obtained from ReceptoPharm, Inc. (Plantation, Fla., USA) was dissolved in saline and was administered intraperitoneally (i.p.) at doses of either 8.5 or 17.0 μg/kg. Enzyme-linked immunosorbent assay (ELISA) kits for detection of the cytokines TNF-α, IL-1, IL-2 and IL-10 were purchased from Boster Biological Technology (Wuhan, China). Complete Freund's Adjuvant (CFA) was purchased from Sigma.

1.3 Rat Model of CFA-induced Adjuvant Arthritis (AA). Male Sprague-Dawley rats were used in this study. Knee joint inflammation was induced by an intra-articular injection of 100 μl CFA. As a control, 100 μl of saline was injected by the same route. Procedures were performed under anesthesia with sodium pentobarbital (50 mg/kg, i.p.). The circumference of the knee joint was measured using a flexible tape measure.

1.4 Administration of Cobratoxin. CTX (8.5 or 17 μg/kg) or the same volume of saline, as control, was administered by i.p injection. To study the effects of CTX on the phase I (first inflammatory reaction, see Example 3 below) in AA rats, CTX (8.5 or 17 μg/kg) or saline was administered three hours prior to the administration of CFA. To study effects of CTX or saline on the phase II response (secondary inflammatory reaction) in AA rats, CTX (8.5 or 17 μg/kg) was administered from 11 to 19 days after the administration of CFA.

1.5 Measurement of Serum Levels of Cytokines. Concentrations of TNF-α, IL-1, IL-2 and IL-10 in serum were determined by ELISA. Rats were anesthetized with sodium pentobarbital (50 mg/kg, i.p.), and blood was collected and stored at −4° C. for 30 min, then centrifuged at 3000×g for 10 min. The supernatant was removed and stored at −20° C. until analysis. Levels of TNF-α, IL-1, IL-2 and IL-10 were determined using enzyme immunoassay kits following the manufacturer's protocol. Measurement was completed using an enzyme-linked immunosorbent assay with an absorbency maximum at 450 nm.

1.6 Assessment of Synovial Membrane Pathology. On the 19^{th} day after adjuvant injection, rats were sacrificed and synovial membranes were removed from the knee joints. Specimens were fixed for six hours in 4% glutaraldehyde in PBS, dissected, decalcified, then placed in a 30% sucrose solution and submitted for routine paraffin embedding. Tissue sections were stained using haematoxylin and eosin (H&E) or Van-Gieson (VG) stain and analyzed by light microscopy.

1.7 Statistical analysis. Unless otherwise stated, data were expressed as mean±standard deviation (S.D.) and evaluated using an analysis of variance (ANOVA) test. Analysis of variance for repeated measurements was used where applicable. The post hoc test was also performed using Student's Newman Keuls test where appropriate. A probability (P) of P<0.05 was considered statistically significant.

### Example 2

### Cobratoxin Reduces Knee Joint Swelling in Animal Model of Arthritis

Groups of rats (n=6 per group) were used in studies to determine the effects of pre- and post-treatment with CTX on knee joint swelling induced by injection of complete Freund's adjuvant in the rat arthritis model described in Example 1 above.

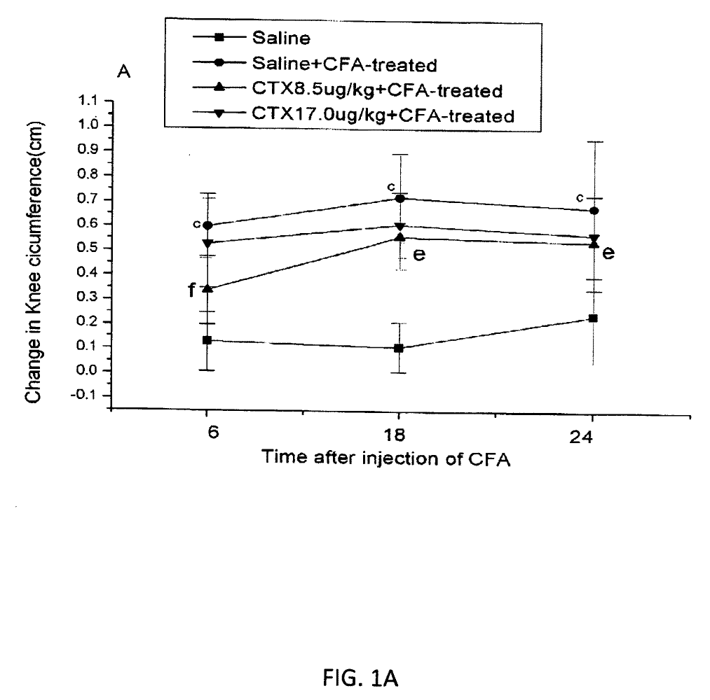

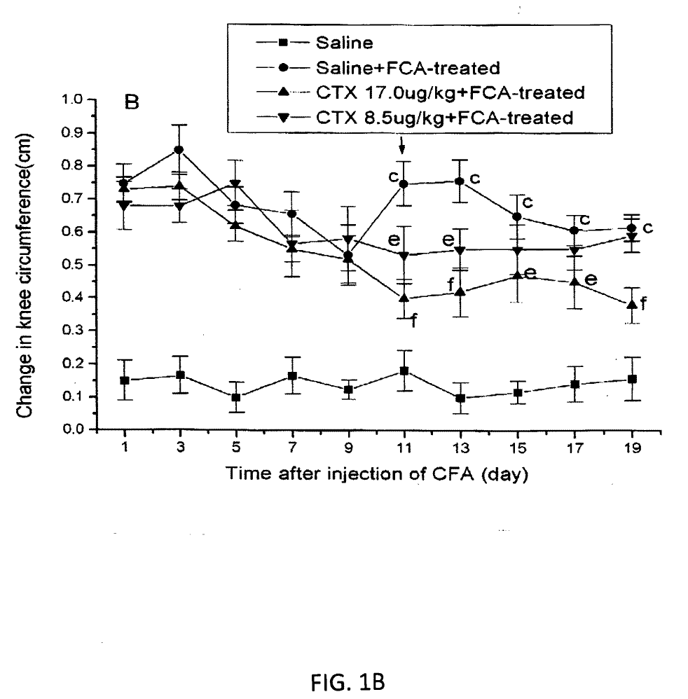

FIGS. 1A and 1B show measurements of the circumference of the knee joint in groups of rats after administration of CFA (to induce AA) or saline (as a control), and further illustrate the effect of administering cobratoxin (CTX) at two dosages (8.5 or 17 μg/kg), either three hours prior to induction of AA with CFA (FIG. 1A, herein termed “pre-treatment”) or at 11 days after AA induction (FIG. 1B, “post-treatment”).

Referring to FIGS. 1A and 1B, it is seen that the circumference of the control (saline-injected) knees remained essentially unchanged over the 19-24 day duration of the experiments. By contrast, the adjuvant-injected knee became and remained swollen for more than 19 days. More particularly, the curve indicated as “Saline+CFA” in FIG. 1B demonstrates that in this animal model of arthritis, the curve of edema (i.e., increase in knee circumference) versus time appears to be divided into two phases. In the first phase (Phase I), the edema increased and reached a peak at 3 days after CFA injection. Subsequently, the edema slowly subsided until the 9th day when the knee began to swell again. The edema peaked on the 11th day and remained elevated thereafter (Phase II).

Referring now to FIG. 1A, pre-treatment with CTX significantly reduced CFA-induced knee edema, as compared with the group treated with CFA+saline. More specifically, in animals pre-treated with CTX, at 6 hr, edema was inhibited by 43.33% and 11.67% respectively, at CTX dosages of 17.0 μg/kg and 8.5 μg/kg. At 18 hr, edema was inhibited by 22.22% and 15.28% respectively, at CTX dosages of 17.0 μg/kg and 8.5 μg/kg.

Similarly, as shown in FIG. 1B, post-treatment with CTX at 11 days after CFA injection significantly reduced inflammation in Phase II of CFA-induced adjuvant arthritis. A statistically significant difference between the CTX- and saline-treated groups was observed from the 11th to the 19th days after CFA administration. The highest inhibition of CFA-induced edema was 46.67% and was observed at the 11th day after CFA (FIG. 1B).

### Example 3

### Cobratoxin Treatment Modulates Levels of Inflammatory Cytokines in Experimental Model of Arthritis

This Example describes studies performed using the rat model of arthritis and immunoassay methods described in Example 1 above to measure the levels of proinflammatory cytokines IL-1, IL-2 and TNF-α in the serum of arthritic animals. The results demonstrate the effect of treatment with cobratoxin on levels of these cytokines.

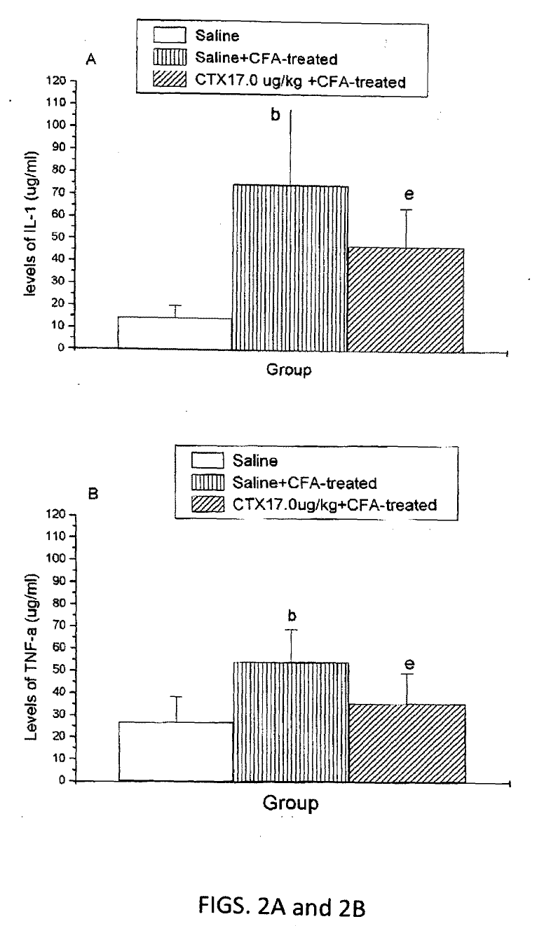

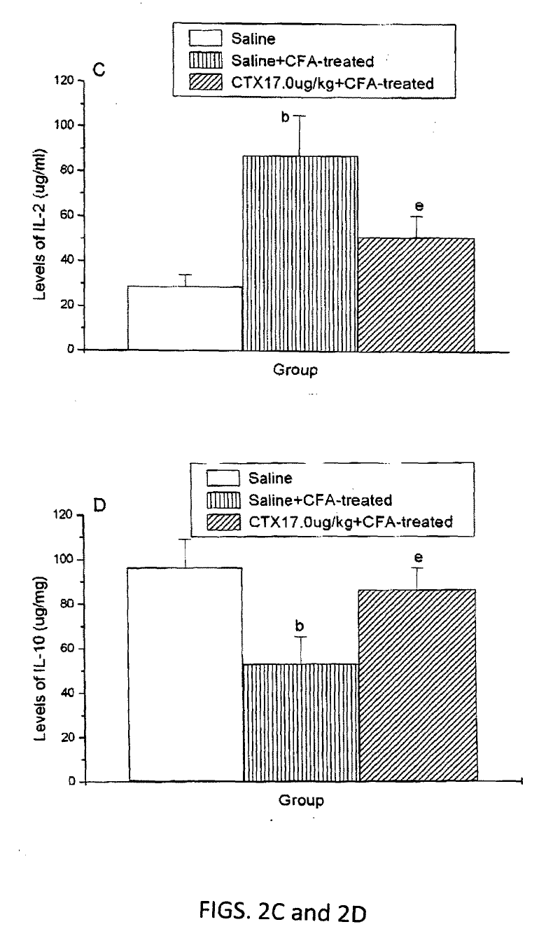

FIG. 2A-2D is a series of four graphs showing levels of IL-1 (FIG. 2A), IL-2 (FIG. 2B), TNF-α (FIG. 2C) and IL-10 (FIG. 2D), as determined by immunoassay under various experimental conditions as described above. The data shown in FIG. 2 are from serum samples assessed on the 19th day after administration of CFA to induce the AA, or of saline in controls. As can be seen by comparing the bars labeled “Saline” and “Saline+CFA-treated” in FIGS. 2A-2C, administration of CFA in the rat model of AA induced a significant increase in the serum concentration of the proinflammatory cytokines IL-1, IL-2 and TNF-α, as compared to the levels in control animals injected with saline alone. By contrast, the serum IL-10 concentration was decreased by CFA administration (FIG. 2D).

In the graphs shown in FIG. 2, the data points represent the mean±S.D (n=6). Symbols indicate probabilities as follows: a P<0.05 and b P<0.01 as compared with Saline group; e P<0.05 f P<0.01 as compared with Saline+CFA group.

Post-treatment with CTX (i.e., 17.0 μg/kg, given i.p. at 11 days after CFA administration) had a marked inhibitory effect on the expression of the proinflammatory cytokines IL-1, IL-2 and TNF-α on 19th day after treatment with CFA (FIGS. 2A-C; compare “CTX 17.0 ug/kg+CFA-treated” with “Saline+CFA-treated”). Conversely, serum IL-10 concentration was significantly increased by CTX, as compared with levels seen in the saline control rats (FIG. 2D).

### Example 4

### Cobratoxin Treatment Reduces Pathophysiological Changes in Synovial Joints in Animal Model of Arthritis

This Example describes the histopathological changes observed in the above-described rat model of arthritis and demonstrates the efficacy of cobratoxin administration for reducing these pathological features.

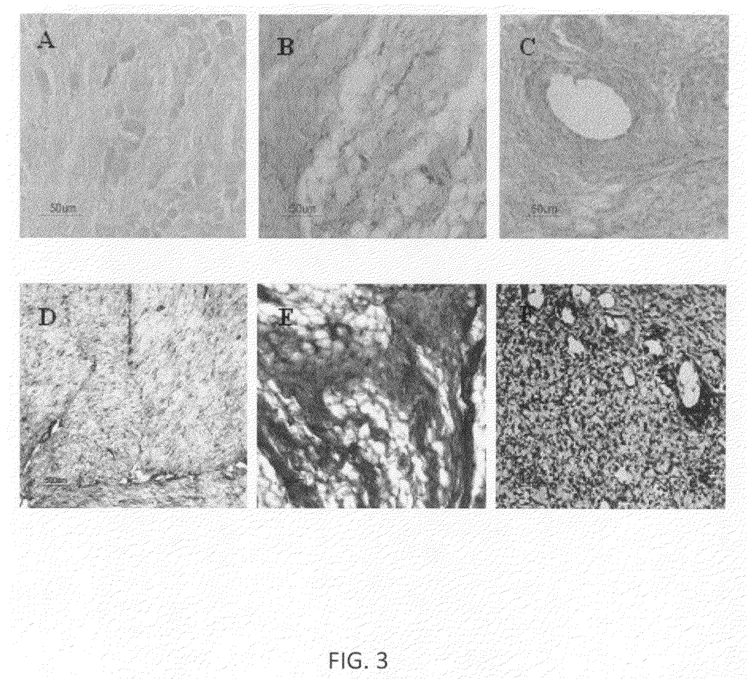

Tissue samples were prepared as described in Example 1 from groups of animals subjected to CFA-induced autoimmune arthritis and saline-administered controls, treated with cobratoxin (CTX) or saline as a control. FIGS. 3A-3F show microscopic images of representative results from these studies. More particularly, the sections shown in FIGS. 3A-C are stained with haematoxylin and eosin (H&E) and those in FIGS. 3D-F are stained with VG stain for visualization of collagen. All images shown in FIGS. 3A-F are shown at a magnification of 400×.

Referring first to FIG. 3A, the histological appearance of normal articular cartilage from the saline-injected group is seen in an H&E-stained paraffin section. Contrasted with this is the appearance of articular cartilage in the Saline+CFA group, showing marked infiltration of inflammatory cells and pannus formation, which is absent in the control group (FIG. 3B; compare with FIG. 3A). The normal appearance of collagen in the articular cartilage, as visualized with the VG stain in a control animal, is shown in FIG. 3D. This is contrasted sharply with the appearance of hyperplastic collagen, as seen in the synovium of an arthritic rat with CFA-induced autoimmune arthritis (compare FIG. 3E with FIG. 3D).

Referring now to FIGS. 3C (H&E stain) and 3F (VG stain), it can be appreciated that the appearance of the articular cartilage in the CFA-treated arthritic rats was greatly improved by treatment with cobratoxin. More particularly, a reduction was seen in the numbers of infiltrating inflammatory cells (FIG. 3C; compare with FIG. 3B). Additionally, the presence of hyperplastic collagen was greatly reduced by the CTX treatment (FIG. 3F; compare with FIG. 3E).

### Example 5

### Topical Treatment of Human Subject with Rheumatoid Arthritis

A 76 year old male with diagnosed rheumatoid arthritis that caused pain and loss of mobility in his hands was treated with a topical pharmaceutical composition that included an α-cobratoxin protein in a cream base, at a concentration of 2 μg of cobratoxin protein per gram of base. Application was on an as needed basis.

The patient observed a decrease in pain, allowing him to feel more comfortable, along with an increase in mobility in the joints in areas to which the therapeutic was applied. The positive effects were achieved within 20 minutes and the relief lasted several hours.

### REFERENCES

It is believed that a review of the following references will increase appreciation of the present invention. The references are referred to throughout the present disclosure by a number as indicated below.
[1] Firestein G S. Evolving concepts of rheumatoid arthritis. Nature 2003; 423: 356-61.
[2] Schwab J H. Bacterial cell-wall induced arthritis: models of chronic recurrent polyarthritid and reactivation of monoarticular arthritis, in: B. Henderson, Edwards J C W, E. R. Pettipher (Eds.), Mechanisms and Models in Rheumatoid Arthritis, Academic Press, San Diego Calif. 1995; 431-46.
[3] Chu C Q, Field M, Feldmann M, Maini R N. Localization of tumor necrosis factor alpha in synovial tissues and at the cartilage-pannus junction in patients with rheumatoid arthritis. Arthritis and Rheumatism 1991; 34: 1125-32.
[4] Pelletier J P, Faure M P, DiBattista J A, Wilhelm S, Visco D, Martel-Pelletier J. Coordinate synthesis of stromelysin, interleukin-1, and oncogene proteins in experimental osteoarthritis. An immunohistochemical study. The American Journal of Pathology 1993; 142: 95-105.
[5] Niki Y, Yamada H, Kikuchi T, Toyama Y, Matsumoto H. Membrane-associated IL-1 contributes to chronic synovitis and cartilage destruction in human IL-1 alpha transgenic mice. J Immunol 2004, 172: 577-84.
[6] Bazzoni F, Beutler B. The tumor necrosis factor ligand and receptor families. New England Journal of Medicine 1996; 334: 1717-25.
[7] Bonecchi R, Bianchi G, Bordignon P P, D'Ambrosio D, Lang R, Borsatti A, et al. Differential expression of chemokine receptors and chemotactic responsiveness of type 1 T helper cells (Th 1 s) and Th2s. Journal of Experimental Medicine 1998; 187: 129-34.
[8] Joosten L A B, Helsen M M A, Saxne T, Van de Loo F A J, Van de Berg W B. IL-1β blockade prevents cartilage and bone destruction in murine type II collagen-induced arthritis, whereas TNFα blockade only ameliorates joint inflammation. The Journal of Immunology 1999; 163: 5049-55.
[9] Newton R C, Decicco C P. Therapeutic potential and strategies for inhibiting tumor necrosis factor-alpha. J Med Chem 1999; 42: 2295-314.
[10] Hirschelmann R, Schade R, Bekemeier H. Acute phase reaction in rats: independent change of acute phase protein plasma concentration and macroscopic inflammation in primary rat adjuvant inflammation. Agents Actions 1990; 30: 412-17.
[11] Nunes F P, Sampaio S C, Santoro M L, Sousa-e-Silva M C. Long-lasting anti-inflammatory properties of Crotalus durissus terrificus snake venom in mice. Toxicon 2007; 49: 1090-98.
[12] Farsky S H, Antunes E, Mello S B. Pro and antiinflammatory properties of toxins from animal venoms. Curr drug targets inflamm allergy 2005; 4: 401-11.
[13] Lukas R J. Diversity and patterns of regulation of nicotinic receptor subtypes. Ann NY Acad Sci 1955; 757: 153-8.
[14] Servent D, Anti-Delbeke S, Gaillard C, Corringer P J, Changeux J P, Menenz A. Molecular characterization of the specificity of interactions of various neurotoxins on two distinct nicotinic acetylcholine receptors. Eur J Pharmacol 2000; 393: 197-204.
[15] Dajas-Bailador F, Costa G, Dajas F, Emmett S. Effects of α-bungarotoxin, α-cobratoxin and fasciculin on the nicotine-evoked release of dopamine in the rat striatum in vivo. Neurochem. Int. 1998; 33: 307-12.
[16] Lena C, Changeux J P. Role of Ca²⁺ ions in nicotinic facilitation of GABA release in mouse thalamus. J Neurosci 1997; 17: 576-85.
[17] Chen Z X, Zhang H L, Gu Z L, Chen B W, Han R, Reid P F, et al. A long-form α-neurotoxin from cobra venom produces potent opioid-independent analgesia. Acta Pharmacologica Sinica 2006; 27: 402-8.
[18] Kaneko M, Tomita T, Nakase T, Ohsawa Y, Seki H, Takeuchi E, et al. Expression of proteinases and inflammatory cytokines in subchondral bone regions in the destructive joint in rheumatoid arthritis. Rheumatology 2001; 40: 247-55.
[19] Arend W P, Dayer J M. Inhibition of the production and effects of interleukin-1 and tumor necrosis factor-α in rheumatoid arthritis. Arthritis Rheumatism 1995; 38: 151-60.
[20] Dayer J M, Fenner H. The role of cytokines and their inhibitors in arthritis. Baillieres Clinical Rheumatology 1992; 6: 485-516.
[21] Moreland L W, Schiff M H, Baumgartner S W, Tindall E A, Fleisch-mann R M, Bulpitt K, et al. Etanercept therapy in rheumatoid arthritis. A randomized, controlled trial. Ann Intern Med 1999; 130, 478-86.
[22] Persson S, Mikulowska A, Narula S, O'Garra A, Holmdahl R. Interleukin-10 suppresses the development of collagen type II-induced arthritis and ameliorates sustained arthritis in rats. Scand. J Immunol 1996; 44: 607-14.
[23] Keystone E, Wherry J, Grint P. IL-10 as a therapeutic strategy in the treatment of rheumatoid arthritis. Rheum. Dis Clin North Am 1998; 24: 629-39.
[24] Katsikis P D, Chu C, Brennan F M, Maini R N, Feldmann M. Immumoregulatory role of interleukin 10 in rheumatoid arthritis. J Exp Med 1994; 179: 1517-27.
[25] Keystone E, Wherry J, Grint P. IL-10 as a therapeutic strategy in the treatment of rheumatoid arthritis. Rheum Dis Clin North Am 1998; 24: 629-39.
[26] Barsante M M, Roffe E, Yokoro C M, Tafuri W L, Souza D G, Pinho V, et al. Anti-inflammatory and analgesic effects of atorvastatin in a rat model of adjuvant-induced arthritis. Euro J Pharm 2005; 516: 282-9.
[27] Pavlov V A, et al. Central muscarinic cholinergic regulation of the systemic inflammatory response during endotoxemia. Proc Nat Acad Sci U.S.A., 2006; 103: 5219-23.
[28] Lawand N B, Lu Y, Westlund K N. Nicotinic cholinergic receptor: potential targets for inflammatory pain relief. Pain 1999; 80: 291-9.
[29] Wang H, et al. Cholinergic agonists inhibit HMGB 1 release and improve survival in experimental sepsis. Nat Med 2004, 10: 1216-21.
[30] Olale F, Gerzanich V, Kuryatov A, Wang F, Lindstrom J. Chronic nicotine exposure differentially affects the function of human a3, a4, and a7 neuronal nicotinic receptor subtypes. JPET 1997; 283: 675-83.
[31] Reitstetter R, Lukas R J, Gruener R. Dependence of nicotinic acetylcholine receptor recovery from desensitization on the duration of agonist exposure. JPET 1999; 289: 656-60.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and each of these documents or references (“herein cited references”), as well as each document or reference cited in each of the herein cited references (including any manufacturer's specifications, instructions, etc.), is hereby expressly incorporated herein by reference.

What is claimed is:

1. A method of treating an arthritic condition, comprising administering to a subject in need thereof an effective amount of a pharmaceutical composition comprising an isolated alpha-neurotoxin protein or an effective variant or fragment thereof.

2. The method of claim 1, wherein the treatment is effective in reducting edema in a joint of said subject.

3. The method of claim 1, wherein the treatment is effective in reducing infiltration of inflammatory cells into articular cartilage in a joint of said subject.

4. The method of claim 1, wherein the treatment is effective in reducing pannus formation in a joint of said subject.

5. The method of claim 1, wherein the treatment is effective in causing a decrease in the level of at least one pro-inflammatory cytokine in the serum of said subject.

6. The method of claim 5, wherein the pro-inflammatory cytokine is selected from the group consisting of TNF-α, interleukin-1 (IL-1), and interleukin-2 (IL-2).

7. The method of claim 1, wherein the treatment is effective in increasing the level of interleukin-10 (IL-10) in the serum of said subject.

8. The method of claim 1, wherein the isolated alpha-neurotoxin protein is derived from the venom of an elapid snake.

9. The method of claim 8, wherein the elapid snake is selected from the group consisting of Naja kaouthia, Naja naja, Naja annulifera, Naja haje, Naja melanoleuca, Naja oxiana and Naja nivea.

10. The method of claim 9, wherein the isolated alpha-neurotoxin protein is a long-form α-cobratoxin protein 71 amino acids in length having the sequence identified as any one of SEQ ID NOS: 1, 2, 3, or 4.

11. The method of claim 1, wherein the isolated alpha-neurotoxin protein is a recombinant protein comprising the amino acid sequence identified as any one of SEQ ID NOS.: 1, 2, 3, or 4, or an effective variant or fragment thereof.

12. The method of claim 1, wherein the isolated alpha-neurotoxin protein is derived from the venom of a snake selected from the group consisting of a Mamba, a King cobra, a Krait and an Australian elapid.

13. The method of claim 1, wherein the composition is administered at a dosage of from 0.01 to 30 micrograms per kilogram of body weight.

14. The method of claim 13, wherein the dosage is from 0.75-8.0 micrograms per kilogram of body weight.

15. The method of claim 1, wherein the composition is administered orally or parenterally.

16. The method of claim 1, wherein the arthritic condition is rheumatoid arthritis.

17. The method of claim 1, wherein the arthritic condition is osteoarthritis.

18. The method of claim 1, wherein the subject is a human.
